(19) United States
(12) Patent Application Publication
(10) Pub. No.: US 2025/0143631 A1
(43) Pub. Date: May 8, 2025

(54) System and Related Methods for AI-driven Skin Condition Diagnosis and Treatment Recommendations

(71) Applicants: Mohsen Khoddami, Mashhad (IR); Maryam Sobhkhiz Sabet, Rasht (IR); RAMI MAMAR, TORONTO (CA); ELHAM SAYYAH, TEHRAN (IR); ELHAM HAFEZI, TEHRAN (IR)

(72) Inventors: Mohsen Khoddami, Mashhad (IR); Maryam Sobhkhiz Sabet, Rasht (IR); RAMI MAMAR, TORONTO (CA); ELHAM SAYYAH, TEHRAN (IR); ELHAM HAFEZI, TEHRAN (IR)

(21) Appl. No.: 18/387,098

(22) Filed: Nov. 6, 2023

(51) Int. Cl. A61B 5/00; G06V 10/74; G16H 30/40; G16H 50/20; G16H 70/60

(52) U.S. Cl. CPC A61B 5/444; A61B 5/7475; G06V 10/74; G16H 30/40; G16H 50/20; G16H 70/60; A61B 5/0077; A61B 5/6898; A61B 5/742; G06V 2201/03

(57) ABSTRACT

The present invention pertains to an AI-Driven Teledermatology Mobile Application for the diagnosis of skin conditions and provision of treatment recommendations. Utilizing artificial intelligence (AI) and machine learning techniques, the application enables users to capture high-resolution images of affected skin areas for analysis. Images are sent to a server equipped with proprietary AI algorithms, which cross-reference them against an extensive dermatological database. A confidence level is calculated for each potential diagnosis. If the confidence level exceeds a predetermined threshold, the application offers treatment suggestions or the option for referral to a certified dermatologist. The system also features an uncertainty check mechanism, an educational library on skin health, and the capacity to retain users' diagnosis history for personalized, future recommendations. The invention aims to provide an efficient and accurate platform for preliminary dermatological consultation, incorporating safety measures for cases of uncertainty or severity.

### FIELD OF INVENTION

The invention pertains to the field of healthcare and telemedicine, specifically focusing on dermatological conditions. It utilizes artificial intelligence (AI) and machine learning algorithms for diagnosing skin conditions via high-resolution images, and provides treatment options as well as referrals to certified dermatologists.

### BACKGROUND

In recent years, the field of dermatology has witnessed significant advancements, particularly in the realm of telemedicine. Dermatological conditions are pervasive, impacting a vast portion of the global populace. While many of these conditions are treatable and often non-life-threatening, the lack of timely and convenient access to specialized care has been a persistent challenge. This challenge is further exacerbated by geographical constraints, financial barriers, and limited availability of expert dermatologists in certain regions.

Teledermatology emerged as a promising solution, offering remote consultations and bridging the gap between patients and dermatologists. However, traditional teledermatology platforms primarily rely on manual consultations, which can lead to extended waiting times and potential delays in diagnosis and treatment. Moreover, the accuracy of diagnosis is inherently dependent on the expertise of the available dermatologist, leading to variability in diagnostic outcomes.

Furthermore, while the prevalence of smartphones and high-resolution cameras has made image capturing more accessible, the interpretation of these images remains a challenge. Existing systems often lack the comprehensive databases required to cross-reference and diagnose a wide array of skin conditions accurately. As a result, patients might receive inconsistent diagnoses based on the same set of images, depending on the consulting dermatologist's experience and the database's comprehensiveness.

Additionally, the vast majority of skin conditions presented to dermatologists are benign and can be treated with over-the-counter remedies or simple lifestyle changes. Yet, the absence of an efficient triaging mechanism in existing platforms means even minor conditions might lead to unnecessary consultations, further straining the already limited dermatological resources.

In light of these limitations, there has been a growing need for an innovative solution that combines the convenience of mobile applications with the power of artificial intelligence. Such a solution should not only facilitate remote consultations but also ensure consistent, accurate, and timely diagnoses, irrespective of the patient's location or the dermatologist's availability. The ultimate objective is to deliver an advanced teledermatology platform that can cater to the diverse needs of the global population while ensuring the highest standards of care and patient satisfaction.

It is within this context that the present invention is provided.

### SUMMARY

The present invention relates to an AI-Driven Mobile Application designed for the diagnosis of skin conditions and the provision of treatment recommendations. This application integrates artificial intelligence (AI) and machine learning algorithms to analyze high-resolution images of skin conditions, subsequently offering treatment suggestions or referrals to certified dermatologists.

In some embodiments, the application provides a user interface that allows users to efficiently capture images of the affected skin area. This interface is tailored to ensure the acquisition of high-quality images suitable for subsequent analysis.

In some embodiments, after the image capture, the images are transmitted to a server equipped with proprietary AI algorithms. These algorithms are tasked with the responsibility of cross-referencing the uploaded images against an extensive dermatological database, allowing for a detailed comparison and analysis.

In some embodiments, the application is designed with an uncertainty check mechanism. If the AI's confidence in its diagnosis is below a predetermined threshold, specifically 50%, the system will generate an error message. This message advises the user to seek professional medical assistance, ensuring a safety net for cases where the AI is uncertain.

In some embodiments, if the AI's confidence level exceeds the 50% threshold, the application will display a percentage-based likelihood of potential skin conditions. Alongside this diagnostic information, the application may also suggest home treatments for conditions deemed non-severe, providing users with immediate guidance.

In some embodiments, the application offers a referral system wherein users can choose to consult a certified dermatologist for a more in-depth diagnosis and potential prescription. This integration ensures that users have access to professional care when required.

In some embodiments, the application encompasses an educational library dedicated to skin health. Users can access this library to obtain comprehensive information on a variety of skin conditions, enhancing their understanding and awareness.

In some embodiments, the application is equipped with a feature that retains the user's diagnosis history. This historical data is utilized to refine subsequent recommendations, thus personalizing the user experience and potentially improving diagnostic accuracy over time.

### BRIEF DESCRIPTION OF THE DRAWINGS

Various embodiments of the invention are disclosed in the following detailed description and accompanying drawings.

FIG. 1 shows a detailed view of an example system architecture for implementing the disclosed invention.

FIG. 2 shows an example process flow of information and decision-making processes initiated when a user uploads an image for diagnostic analysis.

FIG. 3 shows a process flow representing user journey and available functions of a mobile app in an exemplary embodiment of the system.

FIG. 4 shows an example user interface for submitting a dermatological assessment request and its associated data.

Common reference numerals are used throughout the figures and the detailed description to indicate like elements. One skilled in the art will readily recognize that the above figures are examples and that other architectures, modes of operation, orders of operation, and elements/functions can be provided and implemented without departing from the characteristics and features of the invention, as set forth in the claims.

### DETAILED DESCRIPTION AND PREFERRED EMBODIMENT

The following is a detailed description of exemplary embodiments to illustrate the principles of the invention. The embodiments are provided to illustrate aspects of the invention, but the invention is not limited to any embodiment. The scope of the invention encompasses numerous alternatives, modifications and equivalent; it is limited only by the claims.

Numerous specific details are set forth in the following description in order to provide a thorough understanding of the invention. However, the invention may be practiced according to the claims without some or all of these specific details. For the purpose of clarity, technical material that is known in the technical fields related to the invention has not been described in detail so that the invention is not unnecessarily obscured.

### Definitions

The terminology used herein is for the purpose of describing particular embodiments only and is not intended to be limiting of the invention.

As used herein, the term “and/or” includes any combinations of one or more of the associated listed items.

As used herein, the singular forms “a,” “an,” and “the” are intended to include the plural forms as well as the singular forms, unless the context clearly indicates otherwise.

It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof.

The terms “first,” “second,” and the like are used herein to describe various features or elements, but these features or elements should not be limited by these terms. These terms are only used to distinguish one feature or element from another feature or element. Thus, a first feature or element discussed below could be termed a second feature or element, and similarly, a second feature or element discussed below could be termed a first feature or element without departing from the teachings of the present disclosure.

### DESCRIPTION OF DRAWINGS

The present disclosure pertains to a computer-implemented system designed for the diagnosis of dermatological conditions using artificial intelligence (AI) and machine learning techniques. The primary embodiment of this invention is a mobile application that enables users to capture and upload high-resolution images of affected skin areas. These images are then transmitted to a server, which houses a comprehensive dermatological database. The server is equipped with an AI module configured to process the uploaded images and cross-reference them against the database to identify potential matches and diagnose skin conditions.

Upon analyzing the images, the AI module calculates a confidence level for a potential diagnosis based on the identified matches. Depending on this confidence level, the system provides the user with feedback. If the confidence level meets or exceeds a predetermined threshold, the application displays the potential diagnosis and associated percentage-based likelihood to the user. Moreover, the application can suggest home treatments for conditions determined to be non-severe based on the database. If the condition is deemed severe or if the confidence level does not meet the threshold, the user is advised to seek professional medical assistance.

Further, the system integrates a referral module, offering users the option to consult a certified dermatologist directly through the application interface. Additionally, an educational library is incorporated into the mobile application, providing users access to a repository of information related to skin health and specific dermatological conditions.

The aforementioned functionalities are designed to operate in synergy, allowing users to obtain a preliminary diagnosis and treatment suggestions with efficiency and accuracy. Moreover, safety mechanisms are integrated to ensure that in instances of uncertainty or potential severe conditions, users are directed towards professional consultation.

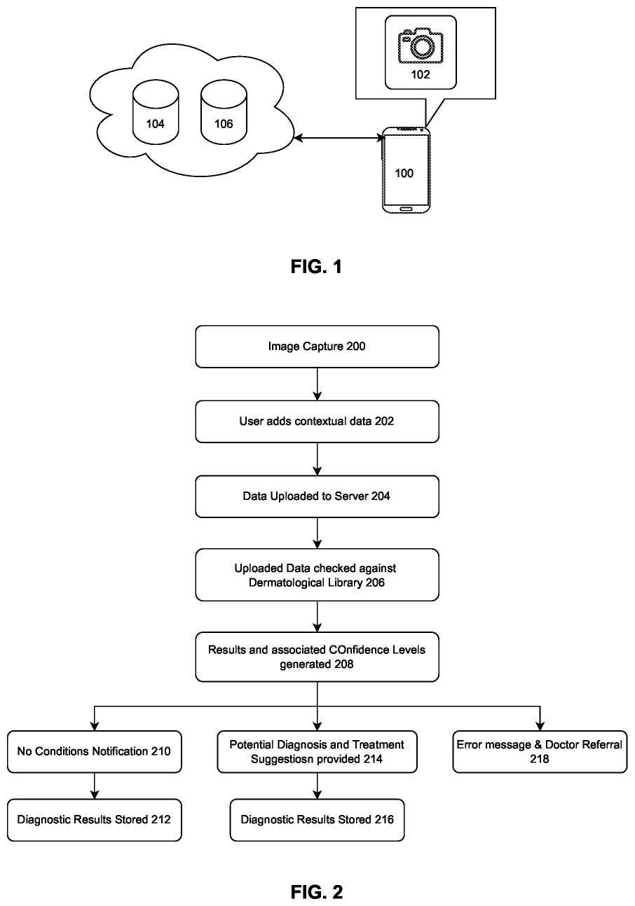

FIG. 1 presents a detailed view of an example system architecture for implementing the disclosed invention, delineating the interconnected components that fulfill the objectives of early skin condition detection, provision of home treatment suggestions, referral options for consultation with certified dermatologists, and overall efficiency in service delivery. The architecture features a mobile device 100 which houses the user interface 102. This interface 102 is equipped with functionalities that guide the user through the process of capturing high-resolution photographs of the affected skin area. Explicit guidelines are presented on the interface 102 to aid the user in acquiring images suitable for diagnostic analysis.

Upon capturing the image, it is transmitted to a cloud server 104 via a secure communication protocol. Residing on this server 104 are proprietary AI algorithms configured to cross-reference the uploaded images with a dermatological database 106. This database 106 is replete with clinical and dermatoscopic data, providing a foundation for the AI's analysis. The server 104 thus serves as the locus for AI-driven diagnosis, housing the computational logic and access to the database 106 that enables this functionality.

Once the AI algorithms complete the analysis, they generate a confidence level for the diagnosis. An uncertainty check mechanism is built into the server 104, where any diagnosis with a confidence level below a predetermined threshold-specifically 50%-triggers an error message. This message is relayed back to the user interface 102, advising the user to seek professional medical assistance.

Conversely, if the confidence level exceeds 50%, the user is presented with diagnostic information on the interface 102. This information includes a percentage-based likelihood of potential skin conditions, derived from the server's analysis. In instances where conditions are deemed non-severe, the interface 102 also provides suggestions for home treatment, fulfilling the objective of immediate user guidance.

Furthermore, the server 104 has the capability to establish a connection with another user device 108 representing a certified dermatologist. The user has the option, via the user interface 102, to book an appointment for a more detailed consultation. The server 104 facilitates this by linking the mobile device 100 and the dermatologist's user device 108.

Supplementing these core features, the system incorporates an educational library, accessible through the user interface 102. This library serves to educate users about various skin conditions, enhancing their awareness and understanding of the diagnostic information presented.

Lastly, the server 104 is configured to store a history of the user's past diagnoses. This historical data is leveraged for refining subsequent treatment recommendations and diagnostic accuracy, thereby contributing to a more personalized user experience over time.

FIG. 2 illustrates an example process flow of information and decision-making processes initiated when a user uploads an image for diagnostic analysis. The sequence commences with the Image Capture step 200, wherein the user takes a photograph of the affected skin area. After the image is captured, the user is prompted to enter additional data regarding their skin disease history 202. This supplementary data serves to enrich the context for the subsequent diagnostic process.

The captured image, along with the entered history data, is then securely transmitted to the cloud server in the Upload to Server step 204. Here, the AI module residing on the server, takes over. It compares the uploaded image and history data to a comprehensive library of dermatological images and associated clinical data 206. The AI module is trained to identify a broad array of skin conditions, ranging from common ailments such as Acne to more serious conditions like Basal Cell Carcinoma.

Following this comparison, the AI module generates a result accompanied by a Confidence Level 208. This confidence level gauges the certainty of the AI's diagnostic outcome and serves as a critical decision-making parameter.

Three possible decision pathways emanate from this point, contingent on the AI result and associated Confidence Level 208:

If the AI assessment indicates that the user has neither a current nor a potential skin condition, and the confidence level surpasses a predetermined threshold, the user is notified via the No Condition Notification step 210. This is followed by the storage of the diagnostic data in the database 212 to preserve a record of the diagnosis.

In cases where one or more disease predictions are generated with a high confidence level above the specified threshold, the process advances to the Potential Diagnosis & Treatment Suggestion step 214. Treatment suggestions, relevant to the identified conditions, are generated and displayed to the user. Subsequently, this diagnosis and treatment data are stored in the database 216 for future reference and analysis.

Alternatively, if the generated confidence level falls below the threshold, the process triggers an Error Message & Doctor Referral step 218. Here, the user is advised that the AI could not arrive at a conclusive diagnosis and is directed to consult a medical professional. A feature within the user interface allows the user to book an appointment, either through video consultation or in person, with a registered doctor.

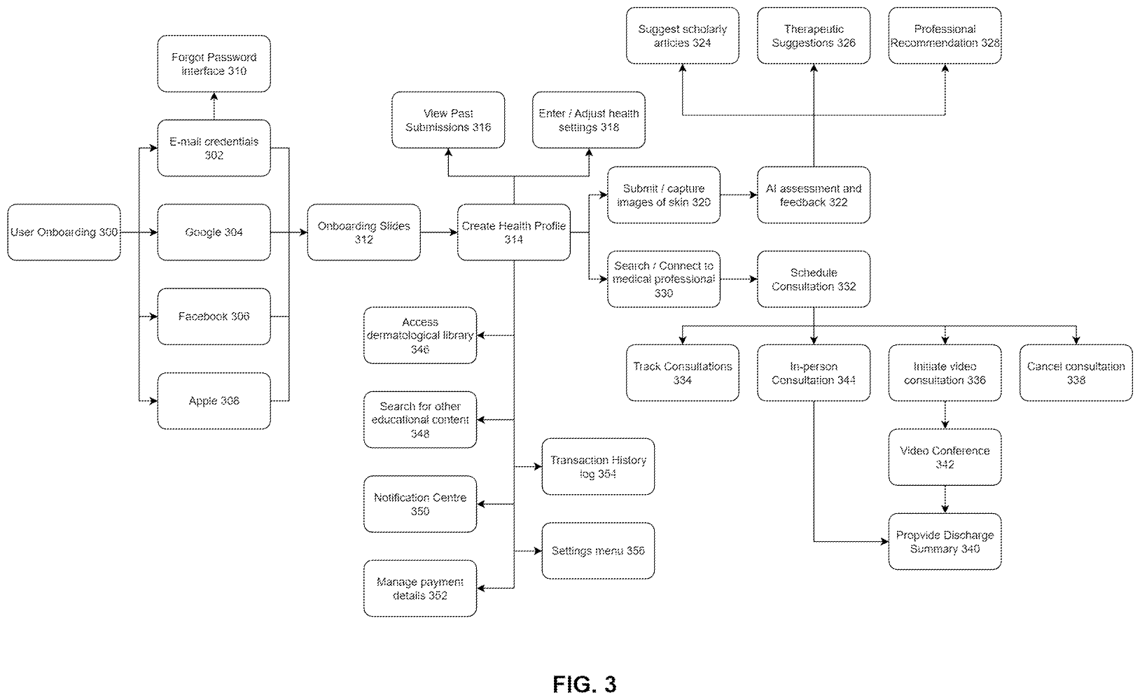

FIG. 3 illustrates a process flow representing user journey and available functions of a mobile app in an exemplary embodiment of the dermatological healthcare application system.

Referring now to FIG. 3, at reference numeral 300, the system initiates the user onboarding process. The system provides several authentication pathways. Users can access the system via established email credentials 302, or through third-party integrations such as Google 304, Facebook 306, or Apple 308. For instances where the user may forget their credentials, the system features a forgotten password interface 310.

Once initial access is secured, the system presents onboarding slides 312 which elucidate the primary capabilities and functionalities available to the user.

Within the confines of the system, users are empowered to manage and formulate their personalized health profiles. At reference numeral 314, a user can create a health profile which may encompass personal metrics such as age, nationality, and gender. The system also permits retrospective viewing of a user's past photo submissions 316 and granular adjustments of their health settings 318.

Central to the system's utility is its image submission and analytical capability. At reference numeral 320, users have the latitude to proffer images either sourced from their device gallery or directly captured via an integrated camera function. Subsequent to image submission, the system, leveraging its proprietary algorithms, renders feedback 322 pertaining to potential dermatological diagnoses. Should the identified condition be amenable to home remedies, the system provisionally supplies relevant scholarly articles 324 and therapeutic suggestions 326. In circumstances requiring more nuanced medical expertise, the system strategically directs users to seek professional intervention 328.

The system also encapsulates functionalities dedicated to physician interaction. Specifically, at reference numeral 330, users can locate medical professionals based on a myriad of criteria including name and geographical locale. The interactive appointment dashboard facilitates the scheduling 332, real-time monitoring 334, initiation 336, or potential cancellation 338 of consultations. Post consultation, the system avails a digitally formatted discharge summary 340. Communication modalities encompass digital video conferencing 342 with integrative capabilities analogous to known platforms such as Google Meet, as well as conventional in-person consultations 344.

A repository of dermatologically-focused articles is accessible to users at reference numeral 346, accompanied by an exhaustive listing 348 of available content.

The system incorporates a sophisticated notification suite at reference numeral 350. This suite permits users to receive and customize notifications germane to the system's offerings and their personal profiles.

Pertaining to financial transactions within the system, reference numeral 352 delineates the payment infrastructure. Noteworthy is the system's design wherein appointments transpiring in a physical context absolve the user from any app-based financial commitments.

To augment user experience and provide comprehensive oversight, the system includes a transaction history log 354 and an assorted collection of ancillary settings 356, encompassing legal terms and conditions, application information, and user support mechanisms.

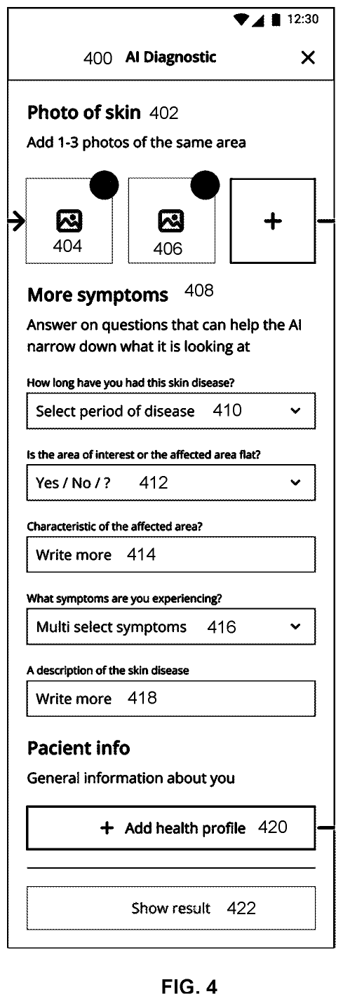

FIG. 4 presents a detailed view of a user interface employed within the dermatological healthcare application system, specifically designated for the capture and submission of user-specific health data, inclusive of visual dermatological evidence.

The header 400 denotes the primary functionality of this interface module, termed “Al Diagnostic”.

Central to the interface is the “Photos of skin” module 402 which invites users to contribute between one to three photographic instances of the dermatological area they wish to assess. Individual frames 404, 406 are provided to preview the uploaded or captured images.

Subsequent to image provision, the system presents a section 408 with options for including additional data about the symptoms.

Within this segment:
Users can specify the duration of the ailment via the “Select period of disease” dropdown, tagged at 410.
Users can specify characteristics of the affected dermatological region, such as dropdown 412 where they can confirm whether the area is flat.
A textual input box at 414 allows them to add other detailed characteristics of the affected area.
Another dropdown list at 416 encourages users to select all experienced symptoms associated with the ailment.
Another textual input area 418 allows input of a free text description of the skin disease.

Subsequently, the “Patient info” segment permits users to integrate and associate their overarching health profile with the current diagnostic request through button 420.

Finally, a command button labeled “Show result” 422 submits the request including all of the entered data for analysis, which once complete, will be returned and displayed on the users mobile device.

### Network Components

A server as described herein can be any suitable type of computer. A computer may be a uniprocessor or multiprocessor machine. Accordingly, a computer may include one or more processors and, thus, the aforementioned computer system may also include one or more processors. Examples of processors include sequential state machines, microprocessors, microcontrollers, graphics processing units (GPUs), central processing units (CPUs), application processors, digital signal processors (DSPs), reduced instruction set computing (RISC) processors, systems on a chip (SoC), baseband processors, field programmable gate arrays (FPGAs), programmable logic devices (PLDs), gated logic, programmable control boards (PCBs), and other suitable hardware configured to perform the various functionality described throughout this disclosure.

Additionally, the computer may include one or more memories. Accordingly, the aforementioned computer systems may include one or more memories. A memory may include a memory storage device or an addressable storage medium which may include, by way of example, random access memory (RAM), static random access memory (SRAM), dynamic random access memory (DRAM), electronically erasable programmable read-only memory (EEPROM), programmable read-only memory (PROM), erasable programmable read-only memory (EPROM), hard disks, floppy disks, laser disk players, digital video disks, compact disks, video tapes, audio tapes, magnetic recording tracks, magnetic tunnel junction (MTJ) memory, optical memory storage, quantum mechanical storage, electronic networks, and/or other devices or technologies used to store electronic content such as programs and data. In particular, the one or more memories may store computer executable instructions that, when executed by the one or more processors, cause the one or more processors to implement the procedures and techniques described herein. The one or more processors may be operably associated with the one or more memories so that the computer executable instructions can be provided to the one or more processors for execution. For example, the one or more processors may be operably associated to the one or more memories through one or more buses. Furthermore, the computer may possess or may be operably associated with input devices (e.g., a keyboard, a keypad, controller, a mouse, a microphone, a touch screen, a sensor) and output devices such as (e.g., a computer screen, printer, or a speaker).

The computer may advantageously be equipped with a network communication device such as a network interface card, a modem, or other network connection device suitable for connecting to one or more networks.

A computer may advantageously contain control logic, or program logic, or other substrate configuration representing data and instructions, which cause the computer to operate in a specific and predefined manner as, described herein. In particular, the computer programs, when executed, enable a control processor to perform and/or cause the performance of features of the present disclosure. The control logic may advantageously be implemented as one or more modules. The modules may advantageously be configured to reside on the computer memory and execute on the one or more processors. The modules include, but are not limited to, software or hardware components that perform certain tasks. Thus, a module may include, by way of example, components, such as, software components, processes, functions, subroutines, procedures, attributes, class components, task components, object-oriented software components, segments of program code, drivers, firmware, micro code, circuitry, data, and/or the like.

The control logic conventionally includes the manipulation of digital bits by the processor and the maintenance of these bits within memory storage devices resident in one or more of the memory storage devices. Such memory storage devices may impose a physical organization upon the collection of stored data bits, which are generally stored by specific electrical or magnetic storage cells.

The control logic generally performs a sequence of computer-executed steps. These steps generally require manipulations of physical quantities. Usually, although not necessarily, these quantities take the form of electrical, magnetic, or optical signals capable of being stored, transferred, combined, compared, or otherwise manipulated. It is conventional for those skilled in the art to refer to these signals as bits, values, elements, symbols, characters, text, terms, numbers, files, or the like. It should be kept in mind, however, that these and some other terms should be associated with appropriate physical quantities for computer operations, and that these terms are merely conventional labels applied to physical quantities that exist within and during operation of the computer based on designed relationships between these physical quantities and the symbolic values they represent.

It should be understood that manipulations within the computer are often referred to in terms of adding, comparing, moving, searching, or the like, which are often associated with manual operations performed by a human operator. It is to be understood that no involvement of the human operator may be necessary, or even desirable. The operations described herein are machine operations performed in conjunction with the human operator or user that interacts with the computer or computers.

It should also be understood that the programs, modules, processes, methods, and the like, described herein are but an exemplary implementation and are not related, or limited, to any particular computer, apparatus, or computer language. Rather, various types of general-purpose computing machines or devices may be used with programs constructed in accordance with some of the teachings described herein. In some embodiments, very specific computing machines, with specific functionality, may be required.

Unless otherwise defined, all terms (including technical terms) used herein have the same meaning as commonly understood by one having ordinary skill in the art to which this invention belongs. It will be further understood that terms, such as those defined in commonly used dictionaries, should be interpreted as having a meaning that is consistent with their meaning in the context of the relevant art and the present disclosure and will not be interpreted in an idealized or overly formal sense unless expressly so defined herein.

The disclosed embodiments are illustrative, not restrictive. While specific configurations of the system have been described in a specific manner referring to the illustrated embodiments, it is understood that the present invention can be applied to a wide variety of solutions which fit within the scope and spirit of the claims. There are many alternative ways of implementing the invention.

It is to be understood that the embodiments of the invention herein described are merely illustrative of the application of the principles of the invention. Reference herein to details of the illustrated embodiments is not intended to limit the scope of the claims, which themselves recite those features regarded as essential to the invention.

What is claimed is:

1. A computer-implemented system for diagnosing skin conditions, comprising
one or more user devices running a mobile application configured to enable a user to capture and upload high-resolution images of an affected skin area;
at least one database having stored thereon a dermatological library comprising a plurality of pre-referenced images and associated skin condition data, and user profiles including previous diagnoses associated with each user profile;
a server in communication with said one or more user devices and the at least one database, said server being configured to run an artificial intelligence (AI) module to:
receive and process high-resolution images from the mobile application;
cross-reference the processed images against the dermatological library to identify potential matches;
calculate a confidence level for a potential diagnosis based on the identified matches; and
transmit the confidence level and potential diagnosis to the requesting user device;

2. The system of claim 1, wherein the mobile application is configured display the potential diagnosis and associated percentage-based likelihood on the mobile application interface if the confidence level is above the predetermined threshold.

3. The system of claim 1, wherein the mobile application is configured to display an error message on if the confidence level is below a predetermined threshold.

4. The system of claim 1, wherein the server is further configured to access said stored user history to refine subsequent diagnostic recommendations.

5. The system of claim 1, wherein the server and mobile application are further configured to implement a referral system to connect users with certified dermatologists upon user request.

6. The system of claim 5, wherein the referral system includes a scheduling module, enabling the user to set up an appointment with a certified dermatologist directly through the mobile application.

7. The system of claim 1, wherein the mobile application interface includes guidelines assisting the user in capturing images, ensuring consistent image quality suitable for Al analysis.

8. The system of claim 1, wherein the AI module is further configured to provide home treatment suggestions for conditions deemed non-severe, based on the cross-referenced analysis against the dermatological database.

9. The system of claim 1, wherein the AI module, upon analyzing the uploaded image, generates a percentage breakdown of probable skin conditions, allowing users to understand the likelihood of each potential diagnosis.

10. The system of claim 1, wherein the AI module is configured to immediately advise users to seek professional medical attention for certain conditions, specifically those with potentially severe implications such as cancer, without suggesting home remedies.

11. The system of claim 1, wherein the at least one database further comprises an educational resource library, accessible from within the mobile application, providing access to information related to various skin conditions.
